(19) United States
(12) Patent Application Publication
(10) Pub. No.: US 2008/0262493 A1
(43) Pub. Date: Oct. 23, 2008

(54) BIPOLAR SCISSORS WITH CURVED SHEAR BLADES

(75) Inventor: Bert Sutter, Freiburg (DE)

Correspondence Address: VOLPE AND KOENIG, P.C., UNITED PLAZA, SUITE 1600, 30 SOUTH 17TH STREET, PHILADELPHIA, PA, 19103, US

(73) Assignee: SUTTER MEDIZINTECHNIK GMBH, Freiburg (DE)

(21) Appl. No.: 12/102,047

(22) Filed: Apr. 14, 2008

(30) Foreign Application Priority Data
Apr. 21, 2007 (DE) 102007018993.3-35

(51) Int. Cl. A61B 18/14

(52) U.S. Cl. 606/49

(57) ABSTRACT

Bipolar scissors (1) with two curved shear blades (2, 3) are provided with insulation on each of the shear blades (2, 3), which is distributed on the two shear blades (2, 3) such that each of the shear blades (2, 3) is insulated only over a part of its length, however the insulation is continuous in this area, i.e. at each of the two shear blades (2, 3) only a single longitudinal area is coated in a lamellar and uninterrupted fashion with insulation (11, 12). The insulation (11, 12) of each of the shear blades (2) complements each other in the closed position of the scissors (11) such that the overall insulation essentially extends over the entire length of the two shear blades (2, 3). This way, each piece of the curved insulation follows the curvature of the shear blade, and in case of mounted insulating plates, there is little stress and the insulation can also withstand several autoclaving processes without separating from the shear blades (2, 3).

### CROSS-REFERENCE TO RELATED APPLICATIONS

This application claims the benefit of DE 10 2007 018 993.3, filed Apr. 21, 2007, which is incorporated herein by reference as if fully set forth.

### BACKGROUND

The invention relates to bipolar scissors with two shear blades provided with two shear planes facing each other and two cutting edges, with the cross-sections of the shear blades extending or being embodied straight in the area of the shear planes up to the cutting edges and the shear blades and thus the shear planes and the cutting edges being laterally curved or bent in the same direction and with insulation being provided in the area of the joint of the shear blades and between the shear blades.

For surgical procedures frequently scissors of various embodiments are used to dissect and/or cut tissue. For dissections the scissors are impressed into the soft structure in a closed state and then opened. Thus the blunt back sides of the shear blades spread the tissue and carefully separate it.

Biological structures, such as vessels or muscle strands, can be uncovered in this manner. Curved or bent shear blades have shown particularly well suited for this purpose. If during such a spreading dissection or during the subsequent sharp cutting using the cutting edges of the shear blades of the scissors a blood vessel is injured or severed in a targeted manner, hemorrhaging occurs. In order to stop the bleeding usually electro-surgical instruments or bipolar tweezers are used. Alternatively the metal scissors can be held to the bleeding vessel stump and simultaneously touched with a mono-polar electrode and electrified. This way the vessel can be closed by way of coagulation.

It is helpful when the scissors are already designed as a coagulation instrument so that during the cutting or immediately thereafter a vessel stub can be coagulated without any additional instruments being fed thereto. Here, it is particularly beneficial when the scissors are embodied as a bipolar instrument, thus electric poles being provided at both shear blades, insulated in reference to each other, so that coagulation can occur even in the proximity of delicate structures without risking any deep damages, as occurring with mono-polar coagulation technology.

Bipolar scissors are known from U.S. Pat. No. 3,651,811 in which not only the shear blades are curved in reference to the operating legs, but additionally the cutting edges extend in a non-straight line, rather they show an arced curvature leading away from each other so that when the scissors are closed the insulation extending only over a partial section of these bipolar scissors of prior art is initially effective, during further closing within the scope of a continuous cutting process, when the blades receding from each other in an arc-shaped manner finally contact, a short-circuit can be expected.

This is avoided in a solution according to U.S. Pat. No. 5,324,289, in which first at least one shear blade is insulated over its entire length, however, here the shear blades and shear planes are shown flat and the cutting edges are embodied extending straight-lined and the insulation produced by coating is subject to strong wear, thus it can lose its effectiveness relatively quickly.

### SUMMARY

Therefore the object is to provide bipolar scissors of the type mentioned at the outset having curved or bent shear blades, which can be produced cost effectively as well as in a robust and stable manner and which is provided with an insulation, stable and durable even in case of frequent use, and having shear blades, relatively thin in their cross-section, and simultaneously allow a simply performed coagulation.

In order to attain this object the bipolar scissors according to the invention are provided such that the shear blades are each insulated only over a part of their length, with the insulations of the two shear blades each being arranged at a single longitudinal area of these shear blades laminar and uninterrupted such that they are offset in reference to each other in the longitudinal direction so that the partial insulation of one shear blade complements the second shear blade in the closed position of the scissors such that the combined insulation formed by the partial insulations essentially extend or is effective over the entire length of the two shear blades.

Although no sufficient insulation is provided over the entire length of either of the shear blades, the insulation is still achieved that is effective over the entire length of the shear blades even in the closed state. Due to the fact that the shear blades are curved, relatively short insulating pieces can be used without any considerable internal stress, while an insulation extending over the entire curvature of the shear blade would be subject to considerable internal stress. This way, even a solid and/or long-lasting insulation can be achieved, particularly in the joint area, where the insulation can be relatively thick without being subject to any disadvantageous stress caused by said curvature.

Here, it is useful that the shear blade insulation in closest proximity of the joint is thicker than that of the second shear blade. Particularly in the area near the joint, the cutting edges of the shear blades pass each other earlier and more frequently so that an insulation arranged in this area thus shows a longer life when it is thicker. However, it is connected to the respective shear blade with little stress, although it is curved, because it can be relatively short.

It is advantageous for an embodiment of the shear blades to be as flat or thin as possible, when at least the insulation arranged near the joint is an insulating plate, inserted into a recess near the joint of the insulated shear blade, made from a material which is as hard as or harder than the metal of the shear blade cooperating therewith. Here, too, it is advantageous that such an insulating plate at the curved shear blade needs to extend over only a part of its overall length such that in spite of the curvature even during sterilization or autoclaving no considerable stress develops, which over time could lead to a separation of the insulating plate from its position of use. A respectively hard insulating material counteracts wear so that long life is achieved and the scissors according to the invention can be used frequently and for a respectively long time.

The insulating plate arranged near the joint can here be a molded part, which is adjusted to the geometry of the shear blade and the joint design as well as possible.

The insulating plate arranged near the joint can be of such thickness that the connection element or the screw for connecting the two shear blades that can be pivoted in reference to each other extends into the insulating plate. In scissors, the joint of the two shear blades is frequently formed by a respectively pin-shaped connection element or a screw, which can be insulated in the same manner by the insulating plate of one of the shear blades.

For example, in order to insulate the connection element or the screw for connecting the two shear blades a sheath can be formed at the insulation, in particularly in one piece in the joint area enwrapping a screw of the end of the pin. Here it is advantageous that a molded piece is used for the insulation in the area near the joint so that it practically has a dual function, but in spite thereof and in spite of the curvature of the shear blades, no unnecessary high stress develops, because it has to extend only over a relatively short partial area of the curved shear blade.

Additionally or instead thereof, a metal screw or a metal pin can be arranged in the joint of the scissors, that is covered or coated with insulation, or the screw or the pin may comprise an insulating material. Here, a sheath formed at the insulation may also form the cover of the metal screw or the metal pin.

The insulating plate or the insulating molded part can be glued, welded, or soldered to the corresponding shear blade. This results in a sufficiently solid, enduring connection.

It is beneficial when the insulation in the insulated areas of the shear blades extend over the entire width of the shear blades. This achieves that even when the scissors are closed entirely no short-circuit can develop.

The distal insulation, distant from the joint, can be arranged at the inner of the two curved shear blades, or perhaps at the exterior shear blade. The arrangement at the inner side of the shear blades joining the interior curve is advantageous here, in that its exterior side is bare, so that the user can operate the scissors very well in a partially opened state for the purpose of coagulation, by facing the tips or ends of the shear blades, pointing in a common direction by the curvature, to the area to be coagulated and to reach it easily and with the blades well insulated against each other. However, when the distal insulated area is provided at the inside of the exterior shear blade only with the relatively large-surface, active back sides of the two shear blades can be coagulated.

The longitudinal insulation at the second shear blade at its area, distant from the joint or distal, can also be made of an insulating inserted plate, a molded part, or an insulating coating. Due to the fact that an insulating plate, based on the division of the two insulations, is relatively short at both shear blades, in spite of the curvature no excessive tension develops, which over time could compromise the connection between the shear blade and the insulation. Therefore, in spite of the curvature of the shear blades, insulating plates can be used, which when arranged on a single shear blade can be subject to considerable tension in the opening direction of an arrangement, which is avoided, however, by the distribution of the insulation onto the two shear blades.

It can be beneficial when complementary insulation of the two shear blades are embodied and arranged at them such and the insulated areas sized such that they slightly overlap in the closed state of the scissors. This ensures that in this transfer area of the two insulations, no short-circuits can occur in a relative position of the shear blades in reference to each other when the scissors are operated.

The length or size of the overlapping area of the two insulations at the two shear blades can range here from approximately one tenth to two millimeters, particularly from one fifth or one fourth or one half or one millimeter or an interim value thereof.

The end of the insulation at the two shear blades can be straight or curved perpendicular to its extension, with the center of the curvature being in the joint of the scissors, and the curved ends of the insulations can extend along a common curvature line or overlap each other.

When the insulation arranged distributed over the two shear blades ends on each side with a curvature, at the curvatures facing each other, with the center of the curvature being the joint of the scissors, at least theoretically overlapping can be omitted because when closing the shear blades the insulations complement each other in any mutual position. However, for safety reasons a slight mutual overlapping might be beneficial in order to allowing for a joint tolerance developing over time, for example.

The longitudinal areas of the shear blades, which are each provided with insulation, can be sized differently at the two shear blades, and particularly the insulation formed by an insulating plate or an insulating molded part in the joint area can be shorter than the insulation of the other shear blade, namely with regard to its portion at the length of the cutting edge. Primarily in the area near the joint, an embodiment of the insulation in the form of an insulating plate or a molded part is particularly beneficial for reasons of wear as well. However, for cost reasons it may be kept shorter than the insulating area of the other shear blade.

The ratio of the two lengths of the insulated longitudinal areas in reference to the cutting edges may amount to approximately one to five or one to four or one to three or one to two or also one to one or an interim value thereof. This can be selected depending on size, cross-section, and width of the shear blades and primarily be adjusted such that the lowermost stress develops in the insulation and at their fastenings to the shear blades.

It is also mentioned that a ceramic part can be provided as an insulating plate or a molded part, which can have an appropriately high solidity and hardness.

It is also mentioned that the described bipolar scissors can be embodied as an open surgical instrument or also as a tubular shaft instrument, as known for example from U.S. Pat. No. 5,540,685 or U.S. Pat. No. 5,352,222, with from the latter publication the common curvature of the two shear blades is known as well.

Primarily in a combination of individual or several of the above-mentioned features and measures bipolar scissors result, either a common open surgical instrument similar to U.S. Pat. No. 5,324,289, or a tubular shaft instrument, which may be robust and stable and yet cheaply produced and which may be provided with a lasting insulation, which is also suitable for frequent utilization without losing its sharpness, even in case of multiple use. However, shear blades with a relatively small thickness can be used, because even when ceramic plates are used as the insulation only little stress develops at the curved shear blades because the insulating ceramic plates extend only over a relatively short longitudinal section of the shear blades so that autoclaving cannot lead to such stress that the curved insulating plates separate from the metallic shear blades. By the distribution of the insulation the radius and/or the curvature to be overcome is only present over a reduced area and thus the stress is lower to a certain extent.

By the use of metal shear blades, here the stability of the instrument remains high with simultaneously showing a relatively small thickness of the shear blades. Additionally, such a design is relatively cost-effective since it is based on conventional prefabricated scissor blanks. Particularly, the use of a ceramic plate for at least one of the two insulating elements at one of the shear blades is advantageous in that it is robust and thus withstands even repeated use and processing. Particularly in the distal area of the instrument, subject to less wear and tear than the joint area, the insulation can also be embodied as a ceramic plate or a molded part, however, here a coating is sufficient for this part.

### BRIEF DESCRIPTION OF THE DRAWINGS

In the following, exemplary embodiments of the invention are described in greater detail using the drawing. Shown in a partially schematic illustration are:

FIG. 1 is a view of bipolar, opened scissors with two shear blades, which are pivotal in reference to a joint via shear arms and handle openings with a view of one of the shear surfaces;

FIG. 2 is a view of the operating end of the scissors with two shear blades and a joint, and in the immediate continuation of the connected shear blades, each single shear blade is shown, with the shear blade in FIGS. 1 and 2 facing away from the observer illustrated in a turned position with regard to the shear surface and its insulation, and the insulation areas at the two shear blades are each limited straight-lined perpendicular in reference to the extension of the shear blades;

FIG. 3 is a view of the two shear blades, with the insulated areas showing an arc-shaped limit perpendicular in reference to the extension of the shear blades and the radius of the curvature of the arc-shaped limit begins at the joint of the scissors and the insulating areas each end in a common limiting line in the operating position;

FIG. 4 is a view similar to FIG. 3, with the insulated areas slightly overlapping each other in the operating position;

FIG. 5 is a top view of the two curved shear blades including the joint area, with the insulation of the area near the joint of the one shear blade and the insulation of the other shear blade, located at the inside, forming a common line with the ends extending perpendicular in reference to the shear blades;

FIG. 6 is a view similar to FIG. 5, with the two insulating areas of the two shear blades slightly overlapping each other;

FIG. 7 is a view similar to FIG. 5, with the insulation near the joint being embodied as a relatively thick insulation plate, which encompasses the end of the screw forming the joint and holding the shear blades together, without the two insulating areas overlapping each other;

FIG. 8 is a view similar to FIG. 6, in which the two insulating areas overlapping each other and the insulation near the joint forming a plate encompassing the end of the connecting screw;

FIG. 9 is a view showing a modified embodiment according to FIG. 7, with the two insulation parts having a common limiting line at their ends extending perpendicular in reference to the shear blades and the insulating plate near the joint having a formed sheath area for encompassing the screw forming the joint;

FIG. 10 is a view similar to FIG. 9 with a sheath part being formed at the insulating plate for encompassing the screw, with the two insulations of the two shear blades slightly overlapping each other in the end areas facing each other;

FIG. 11 is a view similar to FIG. 5, with the distal insulation, in contrast to the embodiment according to FIGS. 5 through 10, not being arranged at the shear blade extending at the interior side of the common curvature but arranged at the shear blade located at the outside and the insulation near the joint being provided at the shear blade, which merges with the curvature located at the inside;

FIG. 12 is a view similar to FIG. 11, with the two insulations of the two shear blades slightly overlapping at an area facing each other;

FIG. 13 is a side view of bipolar scissors according to the invention, embodied as a tubular shaft instrument;

FIG. 14 is, in an enlarged scale, a view of the two shear blades of the instrument according to FIG. 13, with the insulation parts at both shear blades, including the shear blade closer to the observer, being indicated;

FIG. 15 is a top view to the curved shear blades according to FIG. 14, with the insulation near the joint being arranged at the shear blade extending inside along the curvature of the shear blades;

FIG. 16 is a view of the two shear blades according to FIG. 15 in positions separate from each other with the insulation parts each arranged at different longitudinal areas of these shear blades;

FIG. 17 is a view similar to FIG. 14, with the two insulation parts at the two shear blades each being switched in reference to the arrangement of FIG. 14;

FIG. 18 is a top view of the two separately shown shear blades according to FIG. 17, with the insulation being provided at the shear blade extending at the inside of the curvature in its distal area and the insulation of the other shear blade being arranged near the joint and shaped such that it is provided with a sheath-shaped attached part for encompassing the connecting screw;

FIG. 19 is a view similar to FIG. 18, with the insulation near the joint being arranged in the operational state at the curved shear blade extending inside and encompassing the head of the connecting screw in an insulating manner; and

FIG. 20 is a cross-sectional view of the two shearing blades in an area of one of the insulation parts, with the cutting edges just touching each other at this point.

### DETAILED DESCRIPTION OF THE PREFERRED EMBODIMENTS

In the following description of the various embodiments parts identical with regard to their function are marked with identical reference numbers, even in case of modified designs.

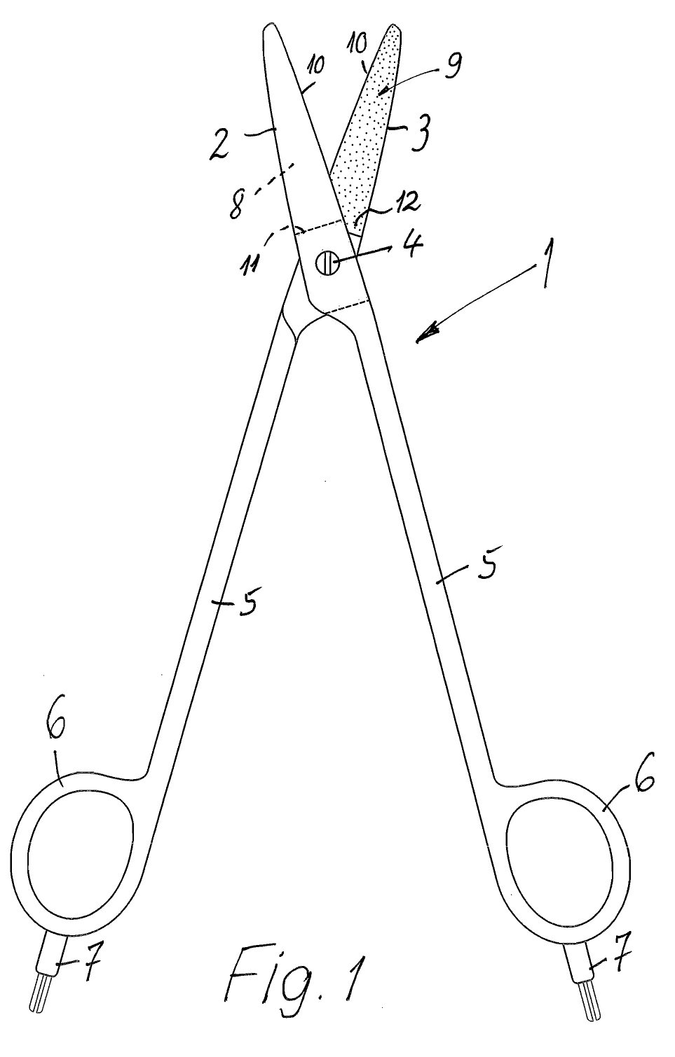

Bipolar scissors marked 1 in their entirety, in the following also called “scissors 1”, can be embodied according to FIGS. 1 through 12 as a surgical instrument for open surgical procedures with two shear blades 2 and 3, which continue over the entire joint 4 to the shear arms 5, at which circular grasping openings 6 are arranged, at which in FIG. 1 the electric connectors 7 are discernible. According to FIGS. 13 through 19 the bipolar scissors 1 can also be a tubular shaft instrument.

In both cases the scissors 1 comprise two shear blades 2 and 3, having shearing planes 8 and 9 and cutting edges 10 facing each other, which is primarily illustrated in FIGS. 2 through 4 and FIG. 20. Here, the shear surface 8 of the shear blades 2 and 3 in FIGS. 1 and 2 in the assembled state of the scissors 1 is not visible, but it is visible in FIG. 2 where the shear blade 2 is turned.

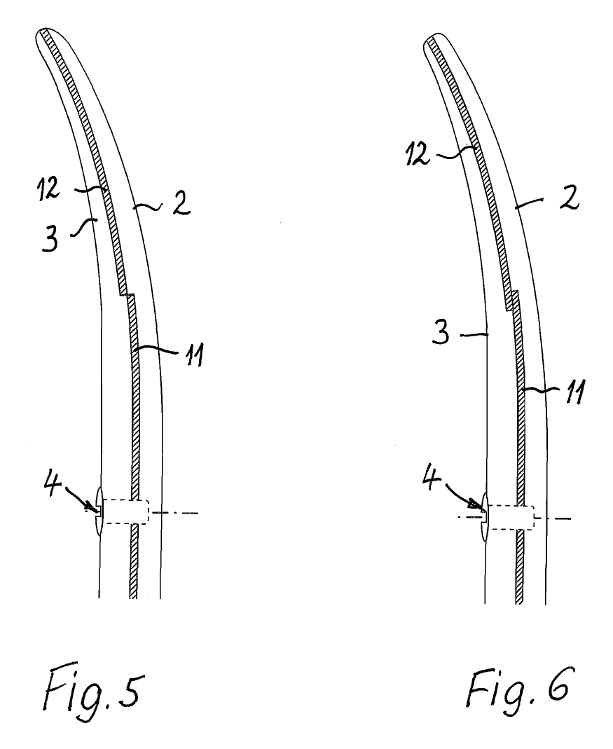

According to FIGS. 5 through 12 and 15 through 19 the shear blades 2 and 3 and the shear surfaces 8 and 9 facing each other as well as the cutting edges 10 are each laterally curved or bent in the same direction, while the cross-sections of the shear blades 2 and 3 at least in the area of the shear surfaces 8 and 9 according to FIG. 20 extend conventionally in a straight line and without any protrusions to the cutting edges 10.

In all of the exemplary embodiments, insulation is provided in a manner to be described in the following in the area of the joint 4 as well as between the shear blades 2 and 3.

Here, in a large number of figures it is discernible, and particularly well in FIGS. 2 through 4 and 15 through 19, that the shear blades 2 and 3 are each insulated only over a portion of their length, but over their entire width, with the insulation 11 and 12 of the two shear blades 2 and 3 extending laminar and uninterrupted over the entire width, and at a single longitudinal section of these shear blades, each insulation part 11, 12 is off-set in the longitudinal direction of the shear blades in reference to each other such that the partial insulation 11 of the first shear blade 2 complements the partial insulation 12 of the second shear blade 3 in the closed position of the scissors according to FIGS. 5 through 12 and 14 through 19 such that the overall insulation formed by the partial insulation parts 11 and 12 essentially extend or are effective over the overall length of the two shear blades 2 and 3 so that even with closed scissors 1, both shear blades 2 and 3 are insulated in reference to each other in spite of the shear blades 2 and 3 each being provided only with a partial insulation 11 and 12, which is advantageous with regard to the straight progression of the cutting edges 10.

While according to FIGS. 5 and 6 and 15 and 16 the insulation 11 and 12 each show approximately the same thickness, the insulation 11 of the insulated shear blade 2 near the joint according to FIGS. 7 through 10 or 18 and 19 may be embodied thicker than the insulation on the other shear blade so that primarily in the area near the joint, where the two shear blades 2 and 3 come into frictional contact with each other more frequently and right from the start at the closing process of the scissors 1, avoiding a disturbing wear of the insulation 11.

Here, in several of the various figures and also in FIGS. 5, 6, 11, and 12 as well as in the other above-mentioned figures concerning the insulation 11 it is discernible that the insulation 11 arranged near the joint is an insulation plate inserted near the joint of the insulated shear blade, which beneficially comprises a material being as hard as or harder than the metal of the cooperating shear blade 2 or perhaps 3.

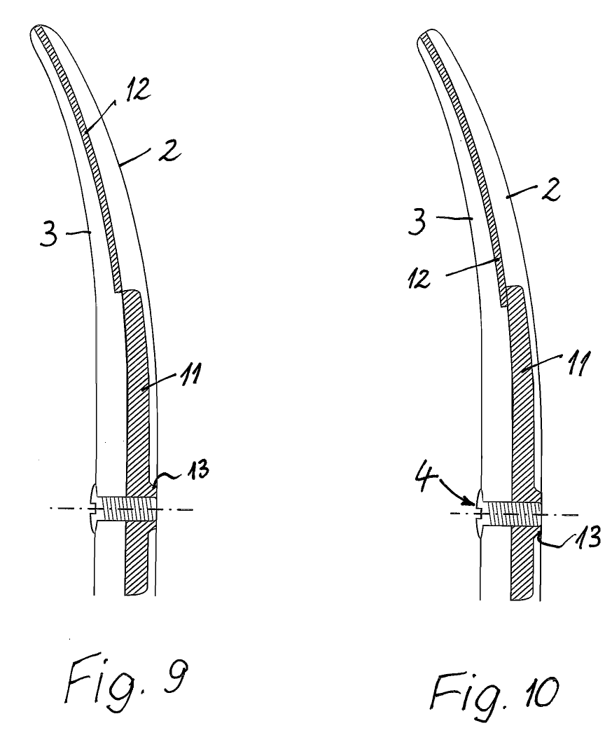

Here, according to FIGS. 9 and 10 as well as 18 and 19, at least the insulating plate near the joint 4, i.e. the insulation 11, is a molded part which on the one hand has a certain cross-sectional thickness and on the other hand also a curving and perhaps a molded piece for the screw allocated to the joint 4.

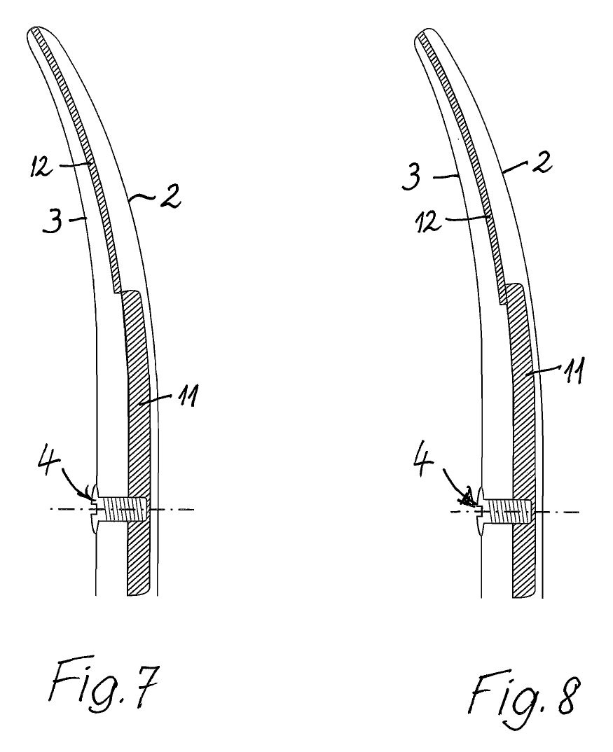

In the exemplary embodiment according to FIGS. 7 and 8, the insulating plate 11 arranged near the joint has a thickness such that the connection element or the screw of the joint 4 for connecting the two shear blades 2 and 3, pivotable in reference to each other, extending into the insulating plate without penetrating it so that the insulating plate of the insulation 11 simultaneously insulates the end of the screw.

In the exemplary embodiments according to FIGS. 9, 10, 18, 19, a sheath 13 is formed in one piece with the insulation encompassing the screw or the end of the pin in order to insulate the connection element or the screw 4 for holding together the two shear blades 2 and 3. Here, the formed sheath 13 is located, according to the exemplary embodiments shown in FIGS. 9, 10, and 18, at the side of the insulation 11 facing away from the other shear blade, because it is arranged at the shear blade 2 extending at the outside of the curved scissors 1.

However, in the exemplary embodiment according to FIG. 19 the formed sheath 13 is located in the area of the screw head, because here the proximal insulation 11 or the one near the joint 4 is arranged at the shear blade 3, which extends at the inside of the curved scissors 1.

Here, in the joint 4 of the scissors 1, a metal screw or a metal pin can be arranged. It could also be coated or covered with insulation or the screw or the pin itself could be made from an insulating material, in order to create good insulation between the two shear blades 2 and 3 in the joint 4 itself.

The insulating plates arranged as insulations 11 and 12 at the shear blades 2 and 3 are here beneficially glued, welded, or soldered to the respective shear blade. Here it is discernible in FIGS. 1 through 4, 14, and 17 that the insulations 11 and 12 in the respectively insulated areas of the shear blades 2 and 3 extend over their entire width so that even when the scissors 1 are closed no short-circuit can occur.

In the exemplary embodiments according to FIGS. 5 through 10 and 18 the distal insulation, distant from the joint 4, is arranged at the interior shear blade 3, which forms the “interior curve” in reference to the exterior shear blade 2. This way, the shear blade 2 and the shear blade 3 spread in case of slightly opened scissors can be held with the ends pointing towards one side due to the curvature to a point to be coagulated, which is therefore well accessible.

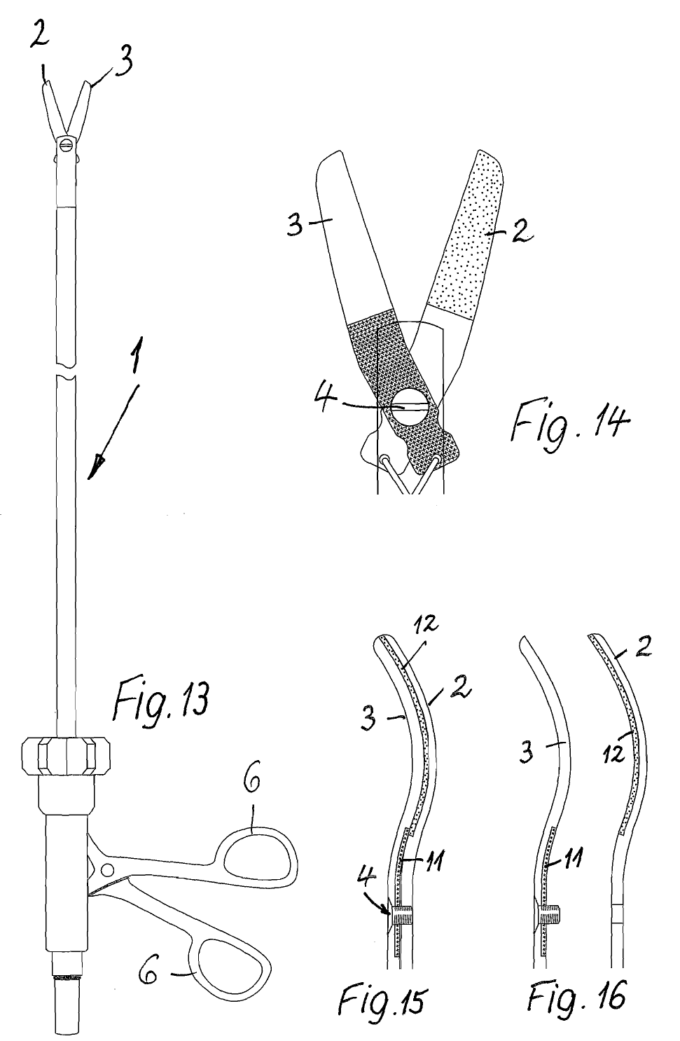

However, it is also possible in an inverse arrangement, in particular in case of a tubular shaft instrument according to FIG. 16, and according to FIGS. 11 and 12 even in case of an instrument for open surgery according to FIG. 1, when the convex side of the shear blades 2 and 3 is preferred for coagulation.

The distal insulation 12, preferably at the second shear blade 3, can also be an insulated plate inserted at its longitudinal area distant from the joint or distal or be embodied as a molded part, or also can be embodied as an insulating coating.

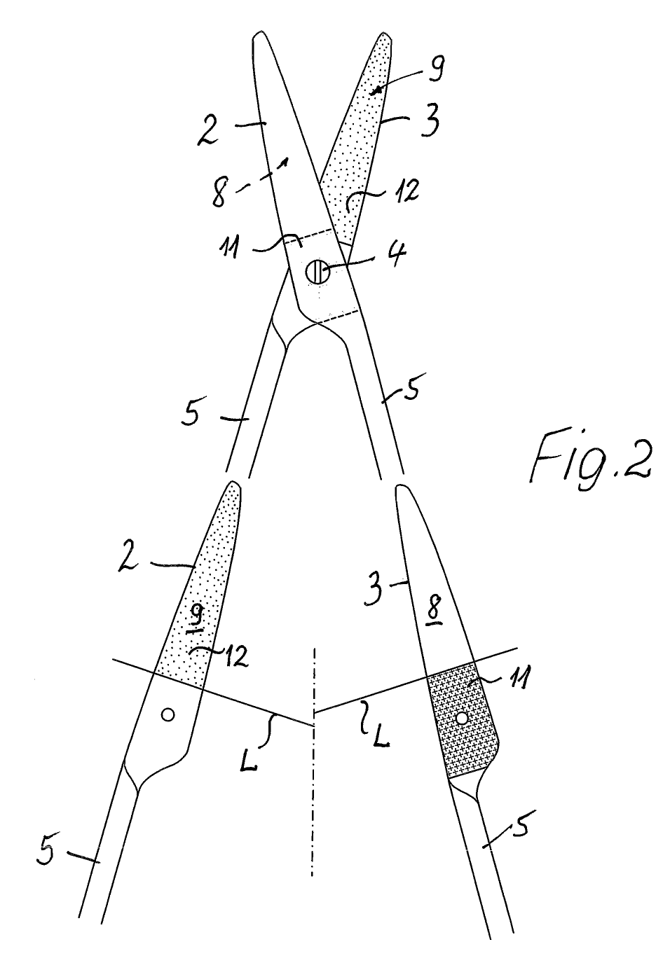

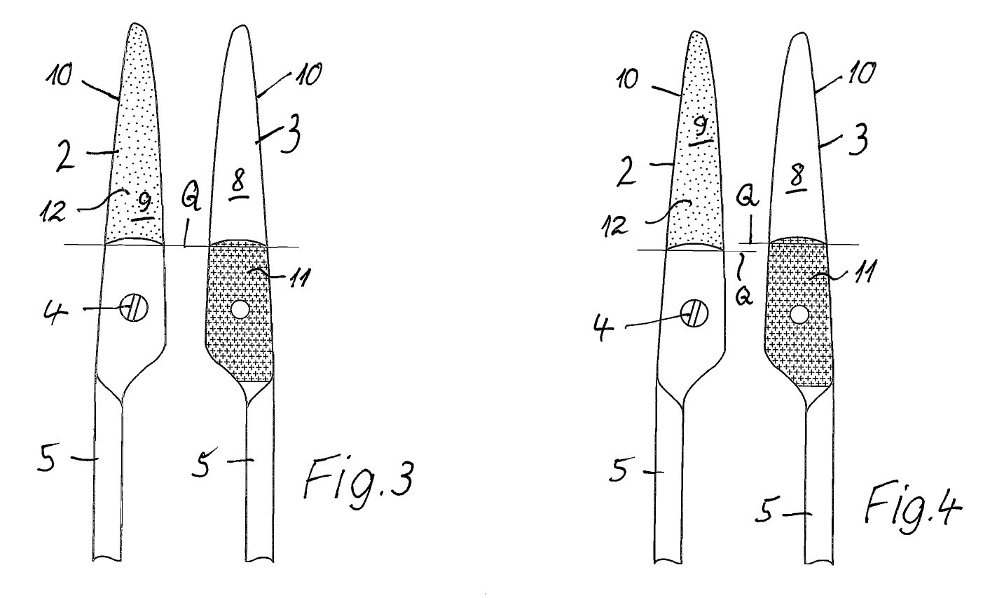

In FIGS. 2 and 4, it is indicated that the complementary insulation parts 11 and 12 of the two shear blades 2 and 3 are embodied and sized here such that they slightly overlap when the scissors 1 are in a closed position. The overlapping area is here illustrated in FIG. 2 by the two lines L slightly off-set at their ends facing each other and in FIG. 4 by the also slightly off-set lateral lines Q. FIG. 3 shows an arrangement in which the overlapping is practically non existent, but the limits of the insulations have or form a common line in the operational position with the lines Q forming a common line.

Primarily in case of a slight overlapping here, the security from any short-circuiting is improved in practically every spread or closed position of the scissors 1, this being the case for both the open instrument according to FIG. 1 as well as the tubular shaft instrument according to FIG. 13.

The length or the size of the overlapping area in the longitudinal direction of the shear blades 2 and 3 can here be extremely small, e.g., amounting to approximately one tenth of a millimeter or less or up to 2 millimeters, in particular approximately one fifth or fourth or one half up to one millimeter, or an interim value between these measurements, which may depend on the measurements of the shear blades 2 and 3 themselves both in the longitudinal as well as the lateral direction and on the cross-sections they show at the exterior sides facing away from the shear surfaces 8 and 9.

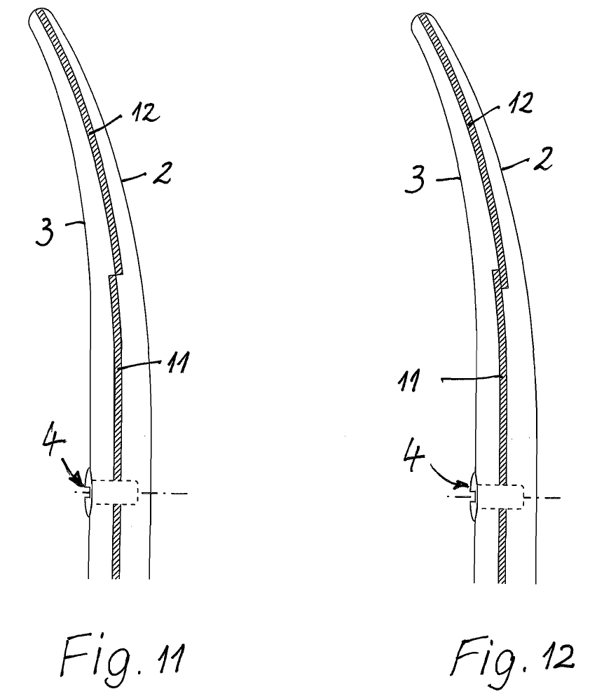

While in the exemplary embodiment according to FIGS. 1 and 2 and according to FIGS. 14 and 17, the limit of the insulation extends perpendicular in a straight line in reference to the shear blades 2 and 3, according to FIGS. 3 and 4 it may also extend curved both in an open instrument according to FIG. 1 as well as in a tubular shaft instrument according to FIG. 13 such that the center of curvature is located at the joint 4 of the scissors 1, so that in a mutual pivoting motion of the shear blades 2 and 3 the mutual overlapping of the insulations 11 and 12 remains unchanged in spite of the pivoting motion, and according to FIG. 3 any overlapping can be even omitted.

In practically all of the exemplary embodiments it is discernible that the longitudinal areas of the shear blades 2 and 3 between their ends and the joint 4, each of which provided with an insulation part 11 and 12, are sized differently at the two shear blades 2 and 3. Primarily the distal insulation 12 is here generally larger than the proximal insulation 11 near the joint, which however has a similar overall length when the area of said insulation 11 extending beyond the joint 4 to the handles 6 is also considered. According to FIG. 1, for example, the insulation near the joint may show only one third of the longitudinal extension of the insulation 12 distant from the joint, when for example the length of the insulations is each compared and measured along the cutting edges 10.

Here, the insulating parts or insulating plates or molded parts may each be ceramic parts, with their hardness withstanding wear and strong resistance.

Bipolar scissors 1 with two curved shear blades 2 and 3 are provided with mutual insulation of the shear blades 2 and 3, which are distributed on the two shear blades 2 and 3 such that each of the shear blades 2 and 3 are only insulated over a portion of their length, however, the insulation is continuous in that part, i.e. at each of the two shear blades 2 and 3 only a single longitudinal section is provided lamellar and uninterrupted with insulation 11 and 12. The insulation 11 and 12 of each of the shear blades 2 complements each other in the closed position of the scissors 1 such that the overall insulation essentially extends over the entire length of the two shear blades 2 and 3. In this way, each of the insulation parts which are bent due to the curvature of the shear blades can be arranged with little stress and in case of insulated plates be mounted and even withstand several autoclaving processes without separating from the shear blades 2 and 3.

It is helpful when the scissors are already designed as a coagulation instrument so that during the cutting or immediately thereafter a vessel stump can be coagulated without any additional instruments being fed thereto. Here, it is particularly beneficial when the scissors are embodied as a bipolar instrument, thus electric poles being provided at both shear blades, insulated in reference to each other, so that coagulation can occur even in the proximity of delicate structures without risking any deep damages, as occurring with mono-polar coagulation technology.

The distal insulation, distant from the joint, can be arranged at the inner of the two curved shear blades, or perhaps at the exterior shear blade. The arrangement at the inner side of the shear blades forming the interior curve is advantageous here, in that its exterior side is bare, so that the user can operate the scissors very well in a partially opened state for the purpose of coagulation, by facing the tips or ends of the shear blades, pointing in a common direction by the curvature, to the area to be coagulated and to reach it easily and with the blades well insulated against each other. However, when the distal insulated area is provided at the inside of the exterior shear blade only with the relatively large-surface, active back sides of the two shear blades can be coagulated.

It is helpful when the scissors are already designed as a coagulation instrument so that during the cutting or immediately thereafter a vessel stump can be coagulated without any additional instruments being fed thereto. Here, it is particularly beneficial when the scissors are embodied as a bipolar instrument, thus electric poles being provided at both shear blades, insulated in reference to each other, so that coagulation can occur even in the proximity of delicate structures without risking any deep damages, as occurring with mono-polar coagulation technology.

The distal insulation, distant from the joint, can be arranged at the inner of the two curved shear blades, or perhaps at the exterior shear blade. The arrangement at the inner side of the shear blades forming the interior curve is advantageous here, in that its exterior side is bare, so that the user can operate the scissors very well in a partially opened state for the purpose of coagulation, by facing the tips or ends of the shear blades, pointing in a common direction by the curvature, to the area to be coagulated and to reach it easily and with the blades well insulated against each other. However, when the distal insulated area is provided at the inside of the exterior shear blade only with the relatively large-surface, active back sides of the two shear blades can be coagulated.

### CROSS-REFERENCE TO RELATED APPLICATIONS

This application claims the benefit of DE 10 2007 018 993.3, filed Apr. 21, 2007, which is incorporated herein by reference as if fully set forth.

### BACKGROUND

The invention relates to bipolar scissors with two shear blades provided with two shear planes facing each other and two cutting edges, with the cross-sections of the shear blades extending or being embodied straight in the area of the shear planes up to the cutting edges and the shear blades and thus the shear planes and the cutting edges being laterally curved or bent in the same direction and with insulation being provided in the area of the joint of the shear blades and between the shear blades.

For surgical procedures frequently scissors of various embodiments are used to dissect and/or cut tissue. For dissections the scissors are impressed into the soft structure in a closed state and then opened. Thus the blunt back sides of the shear blades spread the tissue and carefully separate it.

Biological structures, such as vessels or muscle strands, can be uncovered in this manner. Curved or bent shear blades have shown particularly well suited for this purpose. If during such a spreading dissection or during the subsequent sharp cutting using the cutting edges of the shear blades of the scissors a blood vessel is injured or severed in a targeted manner, hemorrhaging occurs. In order to stop the bleeding usually electro-surgical instruments or bipolar tweezers are used. Alternatively the metal scissors can be held to the bleeding vessel stump and simultaneously touched with a mono-polar electrode and electrified. This way the vessel can be closed by way of coagulation.

It is helpful when the scissors are already designed as a coagulation instrument so that during the cutting or immediately thereafter a vessel stump can be coagulated without any additional instruments being fed thereto. Here, it is particularly beneficial when the scissors are embodied as a bipolar instrument, thus electric poles being provided at both shear blades, insulated in reference to each other, so that coagulation can occur even in the proximity of delicate structures without risking any deep damages, as occurring with mono-polar coagulation technology.

Bipolar scissors are known from U.S. Pat. No. 3,651,811 in which not only the shear blades are curved in reference to the operating legs, but additionally the cutting edges extend in a non-straight line, rather they show an arced curvature leading away from each other so that when the scissors are closed the insulation extending only over a partial section of these bipolar scissors of prior art is initially effective, during further closing within the scope of a continuous cutting process, when the blades receding from each other in an arc-shaped manner finally contact, a short-circuit can be expected.

This is avoided in a solution according to U.S. Pat. No. 5,324,289, in which first at least one shear blade is insulated over its entire length, however, here the shear blades and shear planes are shown flat and the cutting edges are embodied extending straight-lined and the insulation produced by coating is subject to strong wear, thus it can lose its effectiveness relatively quickly.

### SUMMARY

Therefore the object is to provide bipolar scissors of the type mentioned at the outset having curved or bent shear blades, which can be produced cost effectively as well as in a robust and stable manner and which is provided with an insulation, stable and durable even in case of frequent use, and having shear blades, relatively thin in their cross-section, and simultaneously allow a simply performed coagulation.

In order to attain this object the bipolar scissors according to the invention are provided such that the shear blades are each insulated only over a part of their length, with the insulations of the two shear blades each being arranged at a single longitudinal area of these shear blades laminar and uninterrupted such that they are offset in reference to each other in the longitudinal direction so that the partial insulation of one shear blade complements the second shear blade in the closed position of the scissors such that the combined insulation formed by the partial insulations essentially extend or is effective over the entire length of the two shear blades.

Although no sufficient insulation is provided over the entire length of either of the shear blades, the insulation is still achieved that is effective over the entire length of the shear blades even in the closed state. Due to the fact that the shear blades are curved, relatively short insulating pieces can be used without any considerable internal stress, while an insulation extending over the entire curvature of the shear blade would be subject to considerable internal stress. This way, even a solid and/or long-lasting insulation can be achieved, particularly in the joint area, where the insulation can be relatively thick without being subject to any disadvantageous stress caused by said curvature.

Here, it is useful that the shear blade insulation in closest proximity of the joint is thicker than that of the second shear blade. Particularly in the area near the joint, the cutting edges of the shear blades pass each other earlier and more frequently so that an insulation arranged in this area thus shows a longer life when it is thicker. However, it is connected to the respective shear blade with little stress, although it is curved, because it can be relatively short.

It is advantageous for an embodiment of the shear blades to be as flat or thin as possible, when at least the insulation arranged near the joint is an insulating plate, inserted into a recess near the joint of the insulated shear blade, made from a material which is as hard as or harder than the metal of the shear blade cooperating therewith. Here, too, it is advantageous that such an insulating plate at the curved shear blade needs to extend over only a part of its overall length such that in spite of the curvature even during sterilization or autoclaving no considerable stress develops, which over time could lead to a separation of the insulating plate from its position of use. A respectively hard insulating material counteracts wear so that long life is achieved and the scissors according to the invention can be used frequently and for a respectively long time.

The insulating plate arranged near the joint can here be a molded part, which is adjusted to the geometry of the shear blade and the joint design as well as possible.

The insulating plate arranged near the joint can be of such thickness that the connection element or the screw for connecting the two shear blades that can be pivoted in reference to each other extends into the insulating plate. In scissors, the joint of the two shear blades is frequently formed by a respectively pin-shaped connection element or a screw, which can be insulated in the same manner by the insulating plate of one of the shear blades.

For example, in order to insulate the connection element or the screw for connecting the two shear blades a sheath can be formed at the insulation, in particularly in one piece in the joint area enwrapping a screw of the end of the pin. Here it is advantageous that a molded piece is used for the insulation in the area near the joint so that it practically has a dual function, but in spite thereof and in spite of the curvature of the shear blades, no unnecessary high stress develops, because it has to extend only over a relatively short partial area of the curved shear blade.

Additionally or instead thereof, a metal screw or a metal pin can be arranged in the joint of the scissors, that is covered or coated with insulation, or the screw or the pin may comprise an insulating material. Here, a sheath formed at the insulation may also form the cover of the metal screw or the metal pin.

The insulating plate or the insulating molded part can be glued, welded, or soldered to the corresponding shear blade. This results in a sufficiently solid, enduring connection.

It is beneficial when the insulation in the insulated areas of the shear blades extend over the entire width of the shear blades. This achieves that even when the scissors are closed entirely no short-circuit can develop.

The distal insulation, distant from the joint, can be arranged at the inner of the two curved shear blades, or perhaps at the exterior shear blade. The arrangement at the inner side of the shear blades forming the interior curve is advantageous here, in that its exterior side is bare, so that the user can operate the scissors very well in a partially opened state for the purpose of coagulation, by facing the tips or ends of the shear blades, pointing in a common direction by the curvature, to the area to be coagulated and to reach it easily and with the blades well insulated against each other. However, when the distal insulated area is provided at the inside of the exterior shear blade only with the relatively large-surface, active back sides of the two shear blades can be coagulated.

The longitudinal insulation at the second shear blade at its area, distant from the joint or distal, can also be made of an insulating inserted plate, a molded part, or an insulating coating. Due to the fact that an insulating plate, based on the division of the two insulations, is relatively short at both shear blades, in spite of the curvature no excessive tension develops, which over time could compromise the connection between the shear blade and the insulation. Therefore, in spite of the curvature of the shear blades, insulating plates can be used, which when arranged on a single shear blade can be subject to considerable tension in the opening direction of an arrangement, which is avoided, however, by the distribution of the insulation onto the two shear blades.

It can be beneficial when complementary insulation of the two shear blades are embodied and arranged at them such and the insulated areas sized such that they slightly overlap in the closed state of the scissors. This ensures that in this transfer area of the two insulations, no short-circuits can occur in a relative position of the shear blades in reference to each other when the scissors are operated.

The length or size of the overlapping area of the two insulations at the two shear blades can range here from approximately one tenth to two millimeters, particularly from one fifth or one fourth or one half or one millimeter or an interim value thereof.

The end of the insulation at the two shear blades can be straight or curved perpendicular to its extension, with the center of the curvature being in the joint of the scissors, and the curved ends of the insulations can extend along a common curvature line or overlap each other.

When the insulation arranged distributed over the two shear blades ends on each side with a curvature, at the curvatures facing each other, with the center of the curvature being the joint of the scissors, at least theoretically overlapping can be omitted because when closing the shear blades the insulations complement each other in any mutual position. However, for safety reasons a slight mutual overlapping might be beneficial in order to allowing for a joint tolerance developing over time, for example.

The longitudinal areas of the shear blades, which are each provided with insulation, can be sized differently at the two shear blades, and particularly the insulation formed by an insulating plate or an insulating molded part in the joint area can be shorter than the insulation of the other shear blade, namely with regard to its portion at the length of the cutting edge. Primarily in the area near the joint, an embodiment of the insulation in the form of an insulating plate or a molded part is particularly beneficial for reasons of wear as well. However, for cost reasons it may be kept shorter than the insulating area of the other shear blade.

The ratio of the two lengths of the insulated longitudinal areas in reference to the cutting edges may amount to approximately one to five or one to four or one to three or one to two or also one to one or an interim value thereof. This can be selected depending on size, cross-section, and width of the shear blades and primarily be adjusted such that the lowermost stress develops in the insulation and at their fastenings to the shear blades.

It is also mentioned that a ceramic part can be provided as an insulating plate or a molded part, which can have an appropriately high solidity and hardness.

It is also mentioned that the described bipolar scissors can be embodied as an open surgical instrument or also as a tubular shaft instrument, as known for example from U.S. Pat. No. 5,540,685 or U.S. Pat. No. 5,352,222, with from the latter publication the common curvature of the two shear blades is known as well.

Primarily in a combination of individual or several of the above-mentioned features and measures bipolar scissors result, either a common open surgical instrument similar to U.S. Pat. No. 5,324,289, or a tubular shaft instrument, which may be robust and stable and yet cheaply produced and which may be provided with a lasting insulation, which is also suitable for frequent utilization without losing its sharpness, even in case of multiple use. However, shear blades with a relatively small thickness can be used, because even when ceramic plates are used as the insulation only little stress develops at the curved shear blades because the insulating ceramic plates extend only over a relatively short longitudinal section of the shear blades so that autoclaving cannot lead to such stress that the curved insulating plates separate from the metallic shear blades. By the distribution of the insulation the radius and/or the curvature to be overcome is only present over a reduced area and thus the stress is lower to a certain extent.

By the use of metal shear blades, here the stability of the instrument remains high with simultaneously showing a relatively small thickness of the shear blades. Additionally, such a design is relatively cost-effective since it is based on conventional prefabricated scissor blanks. Particularly, the use of a ceramic plate for at least one of the two insulating elements at one of the shear blades is advantageous in that it is robust and thus withstands even repeated use and processing. Particularly in the distal area of the instrument, subject to less wear and tear than the joint area, the insulation can also be embodied as a ceramic plate or a molded part, however, here a coating is sufficient for this part.

### BRIEF DESCRIPTION OF THE DRAWINGS

In the following, exemplary embodiments of the invention are described in greater detail using the drawing. Shown in a partially schematic illustration are:

FIG. 1 is a view of bipolar, opened scissors with two shear blades, which are pivotal in reference to a joint via shear arms and handle openings with a view of one of the shear surfaces;

FIG. 2 is a view of the operating end of the scissors with two shear blades and a joint, and in the immediate continuation of the connected shear blades, each single shear blade is shown, with the shear blade in FIGS. 1 and 2 facing away from the observer illustrated in a turned position with regard to the shear surface and its insulation, and the insulation areas at the two shear blades are each limited straight-lined perpendicular in reference to the extension of the shear blades;

FIG. 3 is a view of the two shear blades, with the insulated areas showing an arc-shaped limit perpendicular in reference to the extension of the shear blades and the radius of the curvature of the arc-shaped limit begins at the joint of the scissors and the insulating areas each end in a common limiting line in the operating position;

FIG. 4 is a view similar to FIG. 3, with the insulated areas slightly overlapping each other in the operating position;

FIG. 5 is a top view of the two curved shear blades including the joint area, with the insulation of the area near the joint of the one shear blade and the insulation of the other shear blade, located at the inside, forming a common line with the ends extending perpendicular in reference to the shear blades;

FIG. 6 is a view similar to FIG. 5, with the two insulating areas of the two shear blades slightly overlapping each other;

FIG. 7 is a view similar to FIG. 5, with the insulation near the joint being embodied as a relatively thick insulation plate, which encompasses the end of the screw forming the joint and holding the shear blades together, without the two insulating areas overlapping each other;

FIG. 8 is a view similar to FIG. 6, in which the two insulating areas overlapping each other and the insulation near the joint forming a plate encompassing the end of the connecting screw;

FIG. 9 is a view showing a modified embodiment according to FIG. 7, with the two insulation parts having a common limiting line at their ends extending perpendicular in reference to the shear blades and the insulating plate near the joint having a formed sheath area for encompassing the screw forming the joint;

FIG. 10 is a view similar to FIG. 9 with a sheath part being formed at the insulating plate for encompassing the screw, with the two insulations of the two shear blades slightly overlapping each other in the end areas facing each other;

FIG. 11 is a view similar to FIG. 5, with the distal insulation, in contrast to the embodiment according to FIGS. 5 through 10, not being arranged at the shear blade extending at the interior side of the common curvature but arranged at the shear blade located at the outside and the insulation near the joint being provided at the shear blade, which merges with the curvature located at the inside;

FIG. 12 is a view similar to FIG. 11, with the two insulations of the two shear blades slightly overlapping at an area facing each other;

FIG. 13 is a side view of bipolar scissors according to the invention, embodied as a tubular shaft instrument;

FIG. 14 is, in an enlarged scale, a view of the two shear blades of the instrument according to FIG. 13, with the insulation parts at both shear blades, including the shear blade closer to the observer, being indicated;

FIG. 15 is a top view to the curved shear blades according to FIG. 14, with the insulation near the joint being arranged at the shear blade extending inside along the curvature of the shear blades;

FIG. 16 is a view of the two shear blades according to FIG. 15 in positions separate from each other with the insulation parts each arranged at different longitudinal areas of these shear blades;

FIG. 17 is a view similar to FIG. 14, with the two insulation parts at the two shear blades each being switched in reference to the arrangement of FIG. 14;

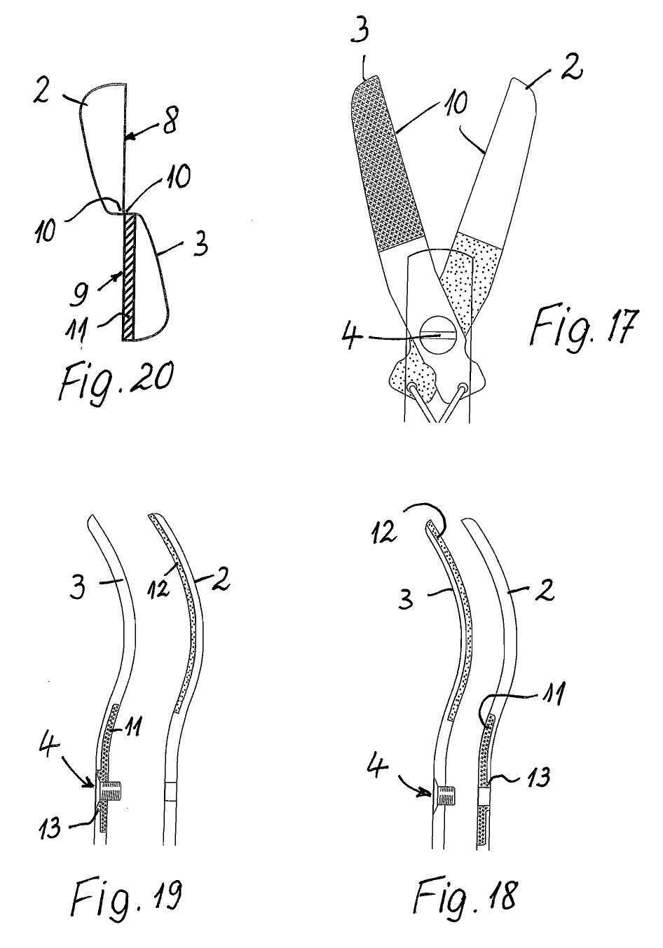

FIG. 18 is a top view of the two separately shown shear blades according to FIG. 17, with the insulation being provided at the shear blade extending at the inside of the curvature in its distal area and the insulation of the other shear blade being arranged near the joint and shaped such that it is provided with a sheath-shaped attached part for encompassing the connecting screw;

FIG. 19 is a view similar to FIG. 18, with the insulation near the joint being arranged in the operational state at the curved shear blade extending inside and encompassing the head of the connecting screw in an insulating manner; and

FIG. 20 is a cross-sectional view of the two shearing blades in an area of one of the insulation parts, with the cutting edges just touching each other at this point.

### DETAILED DESCRIPTION OF THE PREFERRED EMBODIMENTS

In the following description of the various embodiments parts identical with regard to their function are marked with identical reference numbers, even in case of modified designs.

Bipolar scissors marked 1 in their entirety, in the following also called “scissors 1”, can be embodied according to FIGS. 1 through 12 as a surgical instrument for open surgical procedures with two shear blades 2 and 3, which continue over the entire joint 4 to the shear arms 5, at which circular grasping openings 6 are arranged, at which in FIG. 1 the electric connectors 7 are discernible. According to FIGS. 13 through 19 the bipolar scissors 1 can also be a tubular shaft instrument.

In both cases the scissors 1 comprise two shear blades 2 and 3, having shearing planes 8 and 9 and cutting edges 10 facing each other, which is primarily illustrated in FIGS. 2 through 4 and FIG. 20. Here, the shear surface 8 of the shear blades 2 and 3 in FIGS. 1 and 2 in the assembled state of the scissors 1 is not visible, but it is visible in FIG. 2 where the shear blade 2 is turned.

According to FIGS. 5 through 12 and 15 through 19 the shear blades 2 and 3 and the shear surfaces 8 and 9 facing each other as well as the cutting edges 10 are each laterally curved or bent in the same direction, while the cross-sections of the shear blades 2 and 3 at least in the area of the shear surfaces 8 and 9 according to FIG. 20 extend conventionally in a straight line and without any protrusions to the cutting edges 10.

In all of the exemplary embodiments, insulation is provided in a manner to be described in the following in the area of the joint 4 as well as between the shear blades 2 and 3.

Here, in a large number of figures it is discernible, and particularly well in FIGS. 2 through 4 and 15 through 19, that the shear blades 2 and 3 are each insulated only over a portion of their length, but over their entire width, with the insulation 11 and 12 of the two shear blades 2 and 3 extending laminar and uninterrupted over the entire width, and at a single longitudinal section of these shear blades, each insulation part 11, 12 is off-set in the longitudinal direction of the shear blades in reference to each other such that the partial insulation 11 of the first shear blade 2 complements the partial insulation 12 of the second shear blade 3 in the closed position of the scissors according to FIGS. 5 through 12 and 14 through 19 such that the overall insulation formed by the partial insulation parts 11 and 12 essentially extend or are effective over the overall length of the two shear blades 2 and 3 so that even with closed scissors 1, both shear blades 2 and 3 are insulated in reference to each other in spite of the shear blades 2 and 3 each being provided only with a partial insulation 11 and 12, which is advantageous with regard to the straight progression of the cutting edges 10.

While according to FIGS. 5 and 6 and 15 and 16 the insulation 11 and 12 each show approximately the same thickness, the insulation 11 of the insulated shear blade 2 near the joint according to FIGS. 7 through 10 or 18 and 19 may be embodied thicker than the insulation on the other shear blade so that primarily in the area near the joint, where the two shear blades 2 and 3 come into frictional contact with each other more frequently and right from the start at the closing process of the scissors 1, avoiding a disturbing wear of the insulation 11.

Here, in several of the various figures and also in FIGS. 5, 6, 11, and 12 as well as in the other above-mentioned figures concerning the insulation 11 it is discernible that the insulation 11 arranged near the joint is an insulation plate inserted near the joint of the insulated shear blade, which beneficially comprises a material being as hard as or harder than the metal of the cooperating shear blade 2 or perhaps 3.

Here, according to FIGS. 9 and 10 as well as 18 and 19, at least the insulating plate near the joint 4, i.e. the insulation 11, is a molded part which on the one hand has a certain cross-sectional thickness and on the other hand also a curving and perhaps a molded piece for the screw allocated to the joint 4.

In the exemplary embodiment according to FIGS. 7 and 8, the insulating plate 11 arranged near the joint has a thickness such that the connection element or the screw of the joint 4 for connecting the two shear blades 2 and 3, pivotable in reference to each other, extending into the insulating plate without penetrating it so that the insulating plate of the insulation 11 simultaneously insulates the end of the screw.

In the exemplary embodiments according to FIGS. 9, 10, 18, 19, a sheath 13 is formed in one piece with the insulation encompassing the screw or the end of the pin in order to insulate the connection element or the screw 4 for holding together the two shear blades 2 and 3. Here, the formed sheath 13 is located, according to the exemplary embodiments shown in FIGS. 9, 10, and 18, at the side of the insulation 11 facing away from the other shear blade, because it is arranged at the shear blade 2 extending at the outside of the curved scissors 1.

However, in the exemplary embodiment according to FIG. 19 the formed sheath 13 is located in the area of the screw head, because here the proximal insulation 11 or the one near the joint 4 is arranged at the shear blade 3, which extends at the inside of the curved scissors 1.

Here, in the joint 4 of the scissors 1, a metal screw or a metal pin can be arranged. It could also be coated or covered with insulation or the screw or the pin itself could be made from an insulating material, in order to create good insulation between the two shear blades 2 and 3 in the joint 4 itself.

The insulating plates arranged as insulations 11 and 12 at the shear blades 2 and 3 are here beneficially glued, welded, or soldered to the respective shear blade. Here it is discernible in FIGS. 1 through 4, 14, and 17 that the insulations 11 and 12 in the respectively insulated areas of the shear blades 2 and 3 extend over their entire width so that even when the scissors 1 are closed no short-circuit can occur.

In the exemplary embodiments according to FIGS. 5 through 10 and 18 the distal insulation, distant from the joint 4, is arranged at the interior shear blade 3, which forms the “interior curve” in reference to the exterior shear blade 2. This way, the shear blade 2 and the shear blade 3 spread in case of slightly opened scissors can be held with the ends pointing towards one side due to the curvature to a point to be coagulated, which is therefore well accessible.

However, it is also possible in an inverse arrangement, in particular in case of a tubular shaft instrument according to FIG. 16, and according to FIGS. 11 and 12 even in case of an instrument for open surgery according to FIG. 1, when the convex side of the shear blades 2 and 3 is preferred for coagulation.

The distal insulation 12, preferably at the second shear blade 3, can also be an insulated plate inserted at its longitudinal area distant from the joint or distal or be embodied as a molded part, or also can be embodied as an insulating coating. [0075] In FIGS. 2 and 4, it is indicated that the complementary insulation parts 11 and 12 of the two shear blades 2 and 3 are embodied and sized here such that they slightly overlap when the scissors 1 are in a closed position. The overlapping area is here illustrated in FIG. 2 by the two lines L slightly off-set at their ends facing each other and in FIG. 4 by the also slightly off-set lateral lines Q. FIG. 3 shows an arrangement in which the overlapping is practically non existent, but the limits of the insulations have or form a common line in the operational position with the lines Q forming a common line.

Primarily in case of a slight overlapping here, the security from any short-circuiting is improved in practically every spread or closed position of the scissors 1, this being the case for both the open instrument according to FIG. 1 as well as the tubular shaft instrument according to FIG. 13.

The length or the size of the overlapping area in the longitudinal direction of the shear blades 2 and 3 can here be extremely small, e.g., amounting to approximately one tenth of a millimeter or less or up to 2 millimeters, in particular approximately one fifth or fourth or one half up to one millimeter, or an interim value between these measurements, which may depend on the measurements of the shear blades 2 and 3 themselves both in the longitudinal as well as the lateral direction and on the cross-sections they show at the exterior sides facing away from the shear surfaces 8 and 9.

While in the exemplary embodiment according to FIGS. 1 and 2 and according to FIGS. 14 and 17, the limit of the insulation extends perpendicular in a straight line in reference to the shear blades 2 and 3, according to FIGS. 3 and 4 it may also extend curved both in an open instrument according to FIG. 1 as well as in a tubular shaft instrument according to FIG. 13 such that the center of curvature is located at the joint 4 of the scissors 1, so that in a mutual pivoting motion of the shear blades 2 and 3 the mutual overlapping of the insulations 11 and 12 remains unchanged in spite of the pivoting motion, and according to FIG. 3 any overlapping can be even omitted.

In practically all of the exemplary embodiments it is discernible that the longitudinal areas of the shear blades 2 and 3 between their ends and the joint 4, each of which provided with an insulation part 11 and 12, are sized differently at the two shear blades 2 and 3. Primarily the distal insulation 12 is here generally larger than the proximal insulation 11 near the joint, which however has a similar overall length when the area of said insulation 11 extending beyond the joint 4 to the handles 6 is also considered. According to FIG. 1, for example, the insulation near the joint may show only one third of the longitudinal extension of the insulation 12 distant from the joint, when for example the length of the insulations is each compared and measured along the cutting edges 10.

Here, the insulating parts or insulating plates or molded parts may each be ceramic parts, with their hardness withstanding wear and strong resistance.

Bipolar scissors 1 with two curved shear blades 2 and 3 are provided with mutual insulation of the shear blades 2 and 3, which are distributed on the two shear blades 2 and 3 such that each of the shear blades 2 and 3 are only insulated over a portion of their length, however, the insulation is continuous in that part, i.e. at each of the two shear blades 2 and 3 only a single longitudinal section is provided lamellar and uninterrupted with insulation 11 and 12. The insulation 11 and 12 of each of the shear blades 2 complements each other in the closed position of the scissors 1 such that the overall insulation essentially extends over the entire length of the two shear blades 2 and 3. In this way, each of the insulation parts which are bent due to the curvature of the shear blades can be arranged with little stress and in case of insulated plates be mounted and even withstand several autoclaving processes without separating from the shear blades 2 and 3.

1. Bipolar scissors (1) comprising two shear blades (2, 3), provided with shear surfaces (8, 9) facing each other and two cutting edges (10), with cross-sections of the shear blades (2, 3) in an area of the shear surfaces extending or being embodied straight-lined up to the cutting edges (10) and the shear blades and thus the shear surfaces and the cutting edges being curved or bent laterally in the same direction and with insulation being provided in an area of the joint (4) of the shear blades (2, 3) and between the shear blades, the shear blades each being insulated over only a portion of a length thereof, with the insulation (11, 12) of each of the two shear blades (2, 3) being arranged laminar and uninterrupted at a single longitudinal area of the shear blades and off-set in reference to each other in a longitudinal direction of the shear blades such that the insulation (11) extending over a portion of one of the shear blades (2) complements the insulation extending over a portion of the second shear blade (3) in a closed position of the scissors such, that an overall insulation formed by the insulation (11, 12) of each of the shear blades essentially extends or lies effectively over an entire length of the two shear blades (2, 3).

2. Bipolar scissors according to claim 1, wherein the insulation (11) of the shear blade (2) near the joint is thicker than the insulation of the second shear blade.

3. Bipolar scissors according to claim 1, wherein at least the insulation (11) arranged near the joint is inserted into a recess of the shear blade (2) and is made from a material which is as hard or harder than a metal that forms the shear blade it cooperates with.

4. Bipolar scissors according to claim 1, wherein the insulation arranged near the joint comprises a molded insulating plate.

5. Bipolar scissors according to claim 4, wherein the insulating plate (11) arranged near the joint has a thickness that is great enough so that a connecting element or screw for connecting the shear blades (2, 3), pivotable in reference to each other, extends into the insulating plate.

6. Bipolar scissors according to claim 1, wherein for insulating a connecting element or screw for holding the two shear blades (2, 3) together in the joint area (4), a sheath (13) is formed at the insulation (11) encompassing the screw or an end of the connecting element.

7. Bipolar scissors according to claim 1, wherein a metal screw or a metal pin is arranged in the joint (4) of the scissors (1), which is coated or covered with insulation, or the screw or the pin comprises an insulating material.

8. Bipolar scissors according to claim 4, wherein the insulating plate (11) or insulating molded part is glued, welded, or soldered to the shear blade.

9. Bipolar scissors according to claim 1, wherein the insulation in the insulated areas of each of the shear blades (2, 3) extends over an entire width of the shear blades.

10. Bipolar scissors according to claim 1, wherein the insulation (12) that is distal or distant from the joint (4), is arranged on an interior or inner of the two curved shear blades (2, 3) with respect to their curvature.

11. Bipolar scissors according to claim 1, wherein the insulation (12) of the second shear blade (3) at a distal longitudinal area or distant from the joint comprises an insulating inserted plate, a molded part, or an insulating coating.

12. Bipolar scissors according to claim 1, wherein the complementary areas of insulation (11, 12) of each of the two shear blades (2, 3) are each embodied such that the insulating areas are sized such that they slightly overlap in the closed position of the scissors (1).

13. Bipolar scissors according to claim 1, wherein a length or size of the overlapping area of the insulation (11, 12) of each of the two shear blades range from approximately one tenth to two millimeters.

14. Bipolar scissors according to claim 1, wherein a limit of the insulation (11, 12) of each of the two shear blades (2, 3) extends in a straight line or curved, with a center of the curvature being located in the joint (4) of the scissors (1) and the insulation of each of the shear blades limited in a curved manner extends along a common curved line or overlap each other.

15. Bipolar scissors according to claim 1, wherein a longitudinal area of the shear blades (2, 3), each provided with the insulation (11, 12), are differently sized at both of the shear blades (2, 3) and the insulation is formed by an insulating plate or an insulating molded part in the joint area that is shorter in reference to a length of the cutting edge than the insulation of the other shear blade.

16. Bipolar scissors according to claim 1, wherein a ratio of lengths of the insulated longitudinal areas with respect to each other is between approximately one to five to approximately one to one.

17. Bipolar scissors according to claim 4, wherein the insulating plate comprises a ceramic part.

18. Bipolar scissors according to claim 1, wherein the insulation (12) that is distal or distant from the joint (4), is arranged on an exterior or outer of the two curved shear blades (2, 3) with respect to their curvature.
